(19) United States
(12) Patent Application Publication
(10) Pub. No.: US 2023/0279501 A1
(43) Pub. Date: Sep. 7, 2023

(54) Bone Morphogenetic Protein-8B (BMP8B) as marker and therapeutic target for liver fibrosis or liver cancer

(71) Applicant: Friedrich-Alexander-Universität Erlangen-Nürnberg, Erlangen (DE)

(72) Inventor: Claus HELLERBRAND, Erlangen (DE)

(21) Appl. No.: 18/011,569

(22) PCT Filed: Jun. 28, 2021
(86) PCT No.: PCT/EP2021/067637

§ 371 Date: Dec. 20, 2022

(30) Foreign Application Priority Data
Jun. 26, 2020 (DE) 10 2020 207 941.2

(51) Int. Cl. C12Q 1/6886; C12Q 1/6851; C12N 15/113

(52) U.S. Cl. CPC C12Q 1/6886; C12Q 1/6851; C12N 15/1136; C12N 2310/14; C12Q 2600/158; C12Q 2600/106

(57) ABSTRACT

The present invention refers to BMP8B-inhibitor for use in a method of preventing and/or treating liver fibrosis and/or liver cancer, wherein the inhibitor is an oligonucleotide, an antibody or fragment thereof, a small molecule or a combination thereof. Further, the invention is directed to a method for diagnosing liver fibrosis and/or liver cancer and a kit for diagnosing fibrosis and/or cancer. The method and the kit can be used to identify a subject in need for a BMP8B-inhibitor to prevent and/or treat liver fibrosis and/or liver cancer.

The present invention refers to a BMP8B-inhibitor for use in preventing and/or treating liver fibrosis and/or liver cancer, wherein the BMP8B-inhibitor is for example an oligonucleotide, an antibody or fragment thereof, a small molecule or a combination thereof. Further, the present invention is directed to a method for diagnosing liver fibrosis and/or liver cancer and a kit for diagnosing fibrosis and/or cancer.

### TECHNICAL BACKGROUND

Fatty liver disease (FLD), also known as hepatic steatosis, is a condition where excess fat builds up in the liver. Two types of FLD are existing, alcohol-mediated fatty liver disease which is known as alcoholic liver disease and nonalcoholic fatty liver disease (NAFLD).

In its advanced form, FLD is characterized by necroinflammation and progressive fibrosis. Liver fibrosis is the excessive accumulation of extracellular matrix proteins including collagen that occurs in most types of chronic liver disease and can progress to cirrhosis leading to organ failure. Furthermore, cirrhosis is the major risk factor for liver cancer (O'Rourke et al., World J Gastroentrol, 2018).

Liver cancer is one of the most frequent cancers worldwide. It almost exclusively arises in chronic liver disease, and here mostly in the setting of chronic inflammation and fibrosis.

Nonalcoholic fatty liver disease (NAFLD) is one of the most important causes of liver disease worldwide and will probably emerge as the leading cause of end-stage liver disease in the coming decades, with the disease affecting both adults and children.

Usually, NAFLD is associated with obesity and insulin resistance, and therefore, is regarded as the hepatic manifestation of the metabolic syndrome. More than 90% of obese individual have NAFLD.

Hepatocellular fat accumulation is the first pathological step in NAFLD and associated with lipo-toxicity and (oxidative) cellular stress, which result in hepatocellular injury. Hepatic inflammation is frequently triggered by a variety of signals such as pro-inflammatory cytokines and chemokines, released by injured hepatocytes. Furthermore, sustained inflammation and hepatocellular injury are major triggers of hepatic fibrosis (Dietrich, P. & Hellerbrand, C., Best Pract. Res. Clin. Gastroenterol. 2014; Bedossa, P., Liver Int. O. J. Int. Assoc. Study Liver, 2017).

As in other forms of chronic liver disease hepatic fibrosis is the critical pathological step determining morbidity and mortality of NAFLD-patients. However, there is so far no non-invasive method to reliably determine the degree of liver fibrosis.

Still, not all patients progress with advanced fibrosis; however, the only relative accurate diagnosis is invasive and costly liver biopsy. Non-invasive imaging methods are usually neither capable to accurately diagnose liver fibrosis, nor the degree of liver fibrosis progression. Hence, there are no efficient methods available to identify patients at risk for (rapid) fibrosis, which would be important for surveillance programs. Moreover, until now, there is no established medical therapy for the prevention or treatment of NAFLD. It remains that the best available treatment is instructing patients to follow a reasonable dietary plan and a tailored exercise program. Similarly, liver cancer is mostly diagnosed in late stages, i.e., too late for potential curative surgical therapies.

Thus, novel forms of therapies affecting individual pathological steps in NAFLD progression are urgently needed. Furthermore, there is a high medical need for diagnosing methods for early liver fibrosis and/or cancer detection, to identify markers to determine the risk as well as the degree of (already existing) fibrosis and/or liver cancer in patients with NAFLD.

Bone morphogenetic proteins (BMPs) are members of the transforming growth factor ß (TGF-ß) superfamily. BMPs have pleiotropic effects in numerous tissues and physiological processes, in which different BMPs act on specific cell types. BMPs also contribute to liver function in health and disease. Bone morphogenetic proteins (BMPs) are a diverse class of molecules with over 20 growth factor proteins that belong to the transforming growth factor-ß (TGF-ß) family. BMPs are multifunctional cytokines, which were originally discovered for their role in bone and cartilage formation and repair (Wu, M. et al., Bone Res. 2016).

Furthermore, BMPs have been shown to play a critical role in early development, including organogenesis and cell differentiation. More recently, it has been discovered that BMPs are also critically involved in adult homeostasis regulating diverse cellular processes in different organ systems.

Due to the diversity of BMPs, they have different functions which are positive or negative on an organism. BMP9 is for example pro-inflammatory and/or pro-fibrotic, whereas BMP6 is antifibrotic. In the liver, BMPs, but not BMP8B, have been mostly studied in the context of liver regeneration in response to various insults as well as the development and progression of hepatic fibrosis in different pathological conditions. Furthermore, BMPs have been shown to affect systemic energy balance by targeting the pancreas as well as brown and white adipose tissues.

BMP8B is a member of the transforming growth factor ß (TGF-ß) superfamily. BMPs are divided into different subgroups based on their respective amino acid sequence similarity. BMP8B belongs to the osteogenic protein-1 group with BMP5, BMP6, BMP7 and BMP8A as further members. Several studies have indicated an anti-fibrogenic effect of BMP7 in chronic liver disease. Further, BMP6 was shown to be upregulated in steatotic hepatocytes and to counteract activation of primary human hepatic stellate cells and to decrease pro-inflammatory and pro-fibrogenic gene expression of activated HSCs. The role of BMP8B in liver pathophysiology is poorly characterized. BMP8B can activate both canonical as well as non-canonical TGF-beta signaling pathways, which have opposing effects on liver pathology.

It is known that bone morphogenetic protein-8B (BMP8B) is expressed in mature brown adipocytes and uniquely sensitizes brown adipose tissue to adrenergic input, amplifying their thermogenic response (Martins, L., et al., Cell Rep., 2016; Whittle, A. J., et al., Cell, 2012). Its physiological relevance is attested by the reduced thermogenic response and impairment in diet- and cold-induced thermogenesis observed in BMP8B deficient mice (Whittle, A. J., et al., Cell, 2012). Moreover, BMP8B was found to be expressed in the hypothalamus (Contreras, C., et al., Ann. Med., 2015). Central administration of BMP8B induced thermogenesis and increased core temperature, leading to weight loss (Contreras, C., et al., Ann. Med., 2015). Furthermore, BMP8B has been shown to affect the proliferation and maturation of germ-line cells (Zhao, G. Q., et al., Genes Dev., 1996) and there are few studies of BMP8B expression and effects in cancer.

Mahli et al., Cells, 2019 have recently analyzed BMP8B expression in NAFLD and gained first insight into BMP8B effects on the first pathophysiological steps of NAFLD progression, i.e. steatosis and inflammation. Hepatic BMP8B expression was significantly increased in a murine NAFLD model and in NAFLD patients compared with controls. Further, Vacca et al., Nat Metab, 2020 characterized the role of BMP8B in NASH concluding a disease promoting effect of BMP8B in early proinflammatory stages which attenuates significantly in later stages such as liver fibrosis.

As indicated above, so far there is neither a non-invasive, reliable detection of liver fibroses and/or liver cancer available nor a compound which allows effective prevention and/or treatment of liver fibrosis and/or liver cancer having low negative side effects.

The present invention provides BMP8B-inhibitors for effective prevention and/or treatment of liver fibrosis and/or liver cancer. Furthermore, BMP8B represents a biomarker for reliable and accurate diagnosing of liver fibrosis and/or liver cancer as well as monitoring of liver fibrosis and/or liver cancer progression.

### SUMMARY

The present invention refers to a BMP8B-inhibitor for use in a method of preventing and/or treating liver fibrosis and/or liver cancer, wherein the inhibitor is an oligonucleotide, an antibody or fragment thereof, a small molecule or a combination thereof.

The oligonucleotide BMP8B-inhibitor is for example selected from the group consisting of siRNA, miRNA, an antisense oligonucleotide, or a combination thereof. The siRNA, miRNA, or antisense oligonucleotide is for example one type of siRNA, miRNA or antisense oligonucleotide or a pool of siRNAs, a pool of miRNAs, a pool of antisense oligonucleotides, or a combination thereof. The oligonucleotides are for example in the picomolar or nanomolar range.

The BMP8B-inhibitor of the present invention inhibits for example expression and/or activity of the BMP8B specifically in the hepatocyte. It is for example administered locally or systemically for use in preventing and/or treating liver fibrosis; alternatively, the BMP8B-inhibitor of the present invention inhibits expression and/or activity of the BMP8B in the hepatocyte and optionally in other tissue or cell, and e.g., is administered locally or systemically for use in preventing and/or treating liver cancer.

Further, the BMP8B-inhibitor for use according to the present invention inhibits induction of phosphorylation of IkappaB-α and/or p65, the expression of interleukin-8 (IL-8), of intercellular adhesion molecule-1 (ICAM-1), of monocyte chemoattractant protein-1 (MCP-1) or a combination thereof.

The present invention further refers to a pharmaceutical composition comprising a BMP8B-inhibitor of the present invention and a pharmaceutically acceptable carrier.

In addition, the present invention comprises a method for diagnosing liver fibrosis, wherein the concentration of BMP8B is measured in a test sample and the concentration of the BMP8B is compared to the concentration of BMP8B of a healthy control sample, wherein an increase in the BMP8B concentration indicates liver fibrosis.

The increase of the concentration of BMP8B measured in the sample according to the present invention correlates with the degree of the liver fibrosis; for example the increase of the BMP8B concentration distinguishes (i) between stage F0 and F1, F2, F3 or F4, or (ii) between stage F0, F1 or F2 and F3 or F4, or (iii) between stage F0 or F1 and F2, F3 or F4.

The sample of the method for diagnosing liver fibrosis is for example a blood sample or a tissue sample. The technique for measuring the BMP8B concentration in the sample is for example selected from the group consisting of two-dimensional electrophoresis, proteomics, chromatography, Western Blot, immunoblotting, enzyme-linked immunosorbent assay (ELISA) or a combination thereof.

The method for diagnosing liver fibrosis for example comprises measuring of one or more additional marker(s) indicative of liver fibrosis.

The method for diagnosing liver fibrosis is capable of identifying a subject that will benefit from the use of the BMP8B-inhibitor of the present invention and/or to monitor the response of the subject to the use of the BMP8B-inhibitor of the present invention. Moreover, the present invention refers to a kit for diagnosing liver fibrosis or liver cancer in a method of the present invention, wherein the kit comprises for example an antibody or fragment thereof, a probe, a primer or a combination thereof for the detection of the BMP8B concentration. The kit of the present invention optionally comprises additionally the detection of the presence and/or concentration of one or more additional marker(s) indicative of liver fibrosis or liver cancer.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### DESCRIPTION OF FIGURES

FIG. 1A to 1C depict the effect of recombinant BMP8B on the activation of hepatic stellate cells. Primary human hepatic stellate cells (HSC) were isolated and activated by culturing on plastic dishes for 9 days with or without recombinant BMP8B-treatment (doses up to 10 nM). (FIGS. 1A and 1B) α-SMA and COL1A1 mRNA levels analyzed by quantitative RT-PCR. (FIG. 1C) Microscopic phase-contrast images of cultured HSC at day 9. (*: p<0.05 compared with corresponding condition w/o BMP8B stimulation; #: p<0.05 compared with untreated control day 2).

FIGS. 2A and 2B show the effect of recombinant BMP8B on proliferation of activated hepatic stellate cells. Activated human hepatic stellate cells (HSC) were treated with different doses of recombinant BMP8B. (FIG. 2A) Proliferation assessed with the colorimetric XTT assay; (FIG. 2B) phase-contrast images of activated HSC 5 days after incubation. (*: p<0.05 compared to control).

FIG. 3A to 3D depict the effect of recombinant BMP8B on activation of pro-fibrogenic signaling pathways and pro-fibrogenic gene expression in activated hepatic stellate cells. Activated human hepatic stellate cells (HSC) were treated with different doses of recombinant BMP8B. (FIG. 3A) Western blot analysis of p-p65 and p-IκBa protein levels; actin served as control for loading adjustment. (FIG. 3B-3D) Cellular mRNA levels of IL-8, ICAM-1, and MCP-1 analyzed by quantitative RT-PCR. (*: p<0.05 compared to control).

FIGS. 4A and 4B show the BMP8B depletion in steatotic human hepatocytes using siRNA technology. BMP8B expression was depleted in primary human hepatocytes by transfection with siRNA pools directed against BMP8B(si-BMP8B); control cells were transfected with control-siRNA pools (si-Ctr). Subsequently, cells were incubated with or without 0.4 mM oleate (OL) for additional 24 h. (FIG. 4A) BMP8B mRNA levels analyzed by quantitative RT-PCR (FIG. 4B) Western blot analysis of BMP8B protein levels; (*: p<0.05 compared with si-Ctr transfected cells; #: p<0.05 compared with cells w/o oleate stimulation).

FIG. 5 shows that BMP8B secreted from steatotic hepatocytes induces activation of HSC. Primary human hepatic stellate cells (HSC) were isolated and activated by culturing on plastic dishes for 8 days with conditioned media (CM) from BMP8B depleted primary human hepatocytes (CM-Hep^{si-BMP8B}) and control hepatocytes (CM-Hep^{si-Ctr}). α-SMA mRNA levels analyzed by quantitative RT-PCR. α-SMA is a marker for HSC-activation that increases with time. (*: p<0.05).

FIGS. 6A and 6B show significant upregulation of the BMP8B expression in human liver cancer cell lines compared with primary human hepatocytes. (FIG. 6A) BMP8B levels analyzed by quantitative RT-PCR. (FIG. 6B) Western blot analysis of BMP8B protein levels.

FIG. 7 shows significant upregulation of BMP8B expression in human liver cancer tissue compared with non-tumorous liver tissues. Analysis of BMP8B mRNA expression in liver cancer (HCC) and corresponding non-tumorous (NT) tissues of 10 liver cancer patients. BMP8B levels were analyzed by quantitative RT-PCR (*: p<0.05).

FIG. 8 shows that high expression of BMP8B in liver cancer tissues correlates with poor patient survival. Kaplan-Meier survival curve analysis using SurvExpress Biomarker validation database (Aguirre-Gamboa, et al. SurvExpress: An online biomarker validation tool and database for cancer gene expression data using survival analysis; PLoS ONE 2013, 8, e74250.) for a TCGA HCC (LIHC) dataset (n=361) for overall survival with stratification into ‘lowrisk’ and ‘high-risk’ groups based on the prognostic index. The analysis of this dataset revealed a reduced overall survival in liver cancer patients with high BMP8B expression compared to liver cancer patients with low BMP8B expression.

FIG. 9 shows that stimulation with recombinant BMP8B protein induces liver cancer cell proliferation. Human liver cancer cells (HepG2) were stimulated with recombinant BMP8B (10 nM) for 2 days. XTT-assay was used to determine proliferation (*: p<0.05).

FIG. 10 shows that knockdown of BMP8B in liver cancer cells using siRNA directed against BMP8B inhibits liver cancer cell proliferation. Human liver cells (HepG2) were transfected with siRNA directed against BMP8B or Ctr-siRNA. XTT-assay was used to determine proliferation (*: p<0.05).

### DETAILED DESCRIPTION

The present invention identified BMP8B as a biomarker for liver fibrosis and/or liver cancer forming the basis for the prevention and/or treatment of these diseases and/or their diagnosis. Thus, the present invention refers to a BMP8B-inhibitor for use in a method of preventing and/or treating liver fibrosis and/or liver cancer. Further, the present invention is directed to a method for diagnosing liver fibrosis such as chronic liver fibrosis and/or liver cancer, which for example allows identifying a subject that is responsive to the use of a BMP8B-inhibitor for use in a method of preventing and/or treating these diseases.

The present invention surprisingly identified the first time BMP8B as a novel target of inhibition for use in preventing and/or treating liver fibrosis and/or liver cancer, as well as BMP8B as a novel marker for diagnosing liver fibrosis and/or liver cancer by measuring the concentration of BMP8B in a sample.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

The BMP8B-inhibitor of the present invention is for example an oligonucleotide such as siRNA, miRNA, an antisense oligonucleotide or a combination thereof. Oligonucleotide inhibitors provide effective and cost-efficient inhibition of target genes at the post-transcriptional level, avoiding protein production.

An oligonucleotide of the present invention is for example siRNA, miRNA or an antisense nucleotide. It consists of or comprises for example 10 to 30, 12 to 25, 15 to 20, e.g., 10, 11, 12, 13, 14, 15, 16, 17, 18, 19, 20, 21, 22, 23, 24, 25, 26, 27, 28, 29 or 30 nucleotides. The oligonucleotides can further comprise at least one nucleotide which is modified. A modification is for example at least one of sugar-, phosphate backbone-, 5′-Phosphate, base-modifications and combinations thereof. Such modifications may be selected from the group consisting of 2′O-Methyl (2′O-M)-, 2′-Fluoro (2′F)—, 2′-F-arabinonucleic acid (2′FANA)-, 2′O-methoxyethyl (2′O-MOE)-, locked nucleic acid (LNA)-, unlocked nucleic acid (UNA)-, 4′-thioribonucleosides (4'S)—, 4′-C-aminomethyl-2′-O-methyl-, Deoxyribonucleotide (dNMP)-, Cyclohexenyl nucleic acids (CeNA)-, Hexitol nucleic acids (HNA)-, Phosphorothioate (PS)-, Dimethylethylenediamine (DMEDA)-, Amide linker, 5′-C-methyl (S-isomer)-, 5′ (E)-Vinylphosphonate-, 5′ methylenephosphonate-, 2′ thiouridine-, pseudouridine modifications and combinations thereof. The oligonucleotide comprises one or more modifications. The oligonucleotide of the present invention inhibits for example 10 to 99%, 20 to 80%, 30 to 70%, 40 to 60% or 50%, e.g., at least about 40%, 45%, 50%, 55%, 60%, 65%, 70%, 75%, 80%, 85%, 90%, 92%, 94%, 95%, 96%, 97%, 98%, 99%, or 100% of BMP8B expression for example compared to the expression in an untreated subject, tissue or cell. The oligonucleotide-based BMP8B-inhibitor of the present invention is for example a pool of at least two individual oligonucleotides.

siRNA molecules typically cleave target mRNA before translation and have a very tight target specificity. The siRNA molecule of the present invention comprises double-stranded RNA molecules having a polynucleotide sense and a polynucleotide antisense strand. Preferably each strand is for example of 10 to 30, 19 to 25 or 21 to 23 base pair length comprising phosphorylated 5′ ends and hydroxylated 3′ ends with two overhanging nucleotides.

The siRNA molecule of the present invention may further comprise chemical modifications. Chemical modifications of siRNA molecules are for example at least one of sugar-, phosphate backbone-, 5′-Phosphate, base-modifications and combinations thereof. Such modifications may be selected from the group consisting of 2′O-Methyl (2′O-M)-, 2′-Fluoro (2′F)—, 2′-F-arabinonucleic acid (2′FANA)-, 2′O-methoxyethyl (2′O-MOE)-, locked nucleic acid (LNA)-, unlocked nucleic acid (UNA)-, 4′-thioribonucleosides (4′S)—, 4′-C-aminomethyl-2′-O-methyl-, Deoxyribonucleotide (dNMP)-, Cyclohexenyl nucleic acids (CeNA)-, Hexitol nucleic acids (HNA)-, Phosphorothioate (PS)-, Dimethylethylenediamine (DMEDA)-, Amide linker, 5′-C-methyl (S-isomer)-, 5′ (E)-Vinylphosphonate-, 5′ methylenephosphonate-, 2′ thiouridine-, pseudouridine modifications and combinations thereof. siRNA can comprise one or more modifications.

The BMP8B inhibitor of the present invention is a single type of siRNA molecule or a pool of siRNA molecules. The siRNA pools of the present invention comprise high-complexity pools of siRNA molecules for example comprising the most potent siRNA molecules based on thermodynamic properties that favor guide strand loading into the RNA-induced silencing complex (RISC). siRNA pools are designed for maximum coverage of all targeted transcripts with high specificity. They further provide efficient removal of off-target effects by diluting the concentrations of individual siRNA molecules below thresholds that stimulate phenotypes. The siRNA pools of the present invention for example comprise or consist of 50, 40, 30, 20, 10 or 5 individual siRNA molecules. The siRNA molecules or the pool of siRNA molecules are used in picomolar or nanomolar range for example 1 to 100 picomolar, 1 to 50 picomolar, 1 to 10 picomolar, 1 to 100 nanomolar, 1 to 50 nanomolar or 1 to 10 nanomolar such as 1, 2, 3, 4, 5, 6, 7, 8, 9 or 10 nanomolar. Dosages of the siRNA used in the prevention and/or treatment of liver fibrosis and/or liver cancer are for example 0.1 μg/kg body weight to 1 mg/kg body weight such as 0.1 to 0.5 μg/kg body weight.

The term “siRNA” as used herein is intended to include also a precursor of the siRNA which is for example pre-siRNA or pri-siRNA.

The BMP8B-inhibitor of the present invention is alternatively a mircoRNA (miRNA) molecule. miRNA molecules function via base-pairing with complementary or at least partially complementary sequences within the target mRNA. miRNA molecules post-transcriptionally inhibit the target gene by cleavage of the target mRNA, destabilization of the target mRNA or inhibition of the ribosome-mediated protein translation machinery. Preferably miRNA molecules are for example of 10 to 30, 19 to 25 or 21 to 23 base pair length.

The miRNA molecule of the present invention may further comprise for example chemical modifications. Chemical modifications of siRNA molecules are for example at least one of sugar-, phosphate backbone-, 5′-Phosphate, base-modifications and combinations thereof. Such modifications may be selected from the group consisting of 2′O-Methyl (2′O-M)-, 2′-Fluoro (2′F)—, 2′-F-arabinonucleic acid (2′FANA)-, 2′O-methoxyethyl (2′O-MOE)-, locked nucleic acid (LNA)-, unlocked nucleic acid (UNA)-, 4′-thioribonucleosides (4'S)—, 4′-C-aminomethyl-2′-O-methyl-, Deoxyribonucleotide (dNMP)-, Cyclohexenyl nucleic acids (CeNA)-, Hexitol nucleic acids (HNA)-, Phosphorothioate (PS)-, Dimethylethylenediamine (DMEDA)-, Amide linker, 5′-C-methyl (S-isomer)-, 5′ (E)-Vinylphosphonate-, 5′ methylenephosphonate-, 2′ thiouridine-, pseudouridine modifications and combinations thereof. miRNA can comprise one or more modifications.

miRNA molecules are used in a picomolar or nanomolar rangefor example 1 to 100 picomolar, 1 to 50 picomolar, 1 to 10 picomolar, 1 to 100 nanomolar, 1 to 50 nanomolar or 1 to 10 nanomolar such as 1, 2, 3, 4, 5, 6, 7, 8, 9 or 10 nanomolar. Dosages of the miRNA used in the prevention and/or treatment of liver fibrosis and/or liver cancer are for example 0.1 μg/kg body weight to 1 mg/kg body weight such as 0.1 to 0.5 μg/kg body weight. The term “miRNA” as used herein is intended to include also a precursor of the miRNA such as pre-miRNA or pri-miRNA.

The oligonucleotide-based BMP8B-inhibitor of the present invention is for example an antisense oligonucleotide (ASO). The ASO consisting of or comprising preferably 10 to 25 nucleotides, 12 to 20 nucleotides, 11 to 15 nucleotides, 13 to 18 nucleotides, or 14 to 17 nucleotides, e.g., 10, 11, 12, 13, 14, 15, 16, 17, 18, 19, 20, 21, 22, 23, 24, or 25 nucleotides. The ASO may further comprise at least one modified nucleotide. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide or a combination thereof. The ASO of the present invention comprises for example a modified phosphate backbone, wherein the phosphate is for example a phosphorothioate. The ASO are used in a picomolar, nanomolar or micromolar range for example 1 to 100 picomolar, 1 to 50 picomolar, 1 to 10 picomolar, 1 to 100 nanomolar, 1 to 50 nanomolar or 1 to 10 nanomolar such as 1, 2, 3, 4, 5, 6, 7, 8, 9 or 10 nanomolar. Dosages of the antisense oligonucleotide used in the prevention and/or treatment of liver fibrosis and/or liver cancer are for example 0.1 μg/kg body weight to 1 mg/kg body weight such as 0.1 to 0.5 μg/kg body weight

Alternatively, the BMP8B-inhibitor of the present invention is for example an antibody or a fragment thereof.

An antibody or a fragment thereof is a protein or polypeptide which binds specifically to a particular antigen such as BMP8B. Antibodies of the present invention for example consist of five main classes of IgA, IgD, IgE, IgG and IgM. The antibody Fe domain for example comprises or consists of an Fe domain of the IgA, IgM, IgE, IgD or IgG antibody, or a variant thereof. The CDRs of the antibody or fragment thereof for example comprises or consists of CDR1, CDR2, CDR3 or variants thereof. The antibodies of the present invention are for example monoclonal or polyclonal antibodies.

BMP8B belongs to the osteogenic protein-1 group with BMP5, BMP6, BMP7 and BMP8A as further members. BMP8B shows significant homology with these other members, e.g., 74% amino acid sequence homology to BMP7. BMP8B and BMP8A have even 98.3% amino acid sequence homology. BMP8B protein is processed into a 139-amino acid mature form. For serum analysis, it is for example necessary to detect the mature BMP8B form. The mature BMP8B form differs in only 3 amino acids (aa) from BMP8A (aa293, aa 358 and aa 360). Thus, polypeptides including the area aa293 and/or 358 and aa 360 need to be used for the generation of (monoclonal) antibodies for the specific detection of BMP8B and differentiation from BMP8A, respectively. To differentiate BMP8B (and BMP8A) from the other family members (BMP5, BMP6, BMP7) for example the region aa 265 to aa 297 is used. Thus, polypeptides including parts of the region aa 265 to aa 297 are for example used for the generation of monoclonal and/or polyclonal antibodies for the specific detection of BMP8B/A and differentiation from BMP5, BMP6 and/or BMP7.

For the specific detection of BMP8B in the serum for example a sandwich ELISA is used comprising (i) a monoclonal and/or polyclonal antibody directed against a polypeptide containing parts of the region aa 265 to aa 297 as capture and detection antibodies or (ii) a monoclonal and/or polyclonal antibody directed against a polypeptide containing parts of the region around the area aa293 and/or 358 and aa 360 for example in combination with a monoclonal and/or polyclonal antibody directed against a polypeptide containing parts of the region aa 265 to aa 297 as capture and detection antibodies.

The BMP8B-inhibitor of the present invention is for example a small molecule. Small molecules comprise low-molecular weight (e.g., <900 daltons) organic compounds. Small molecules for example bind specific macromolecules and act as effectors, altering function or activity of the target. Small molecules are easy and cheap to manufacture due to the small size. This allows small molecules to translocate through the plasma membrane and interact with the cytoplasmic domain of cell-surface receptors and intracellular signaling molecules.

The small molecule of the present invention binds for example BMP8B or factors activated by BMP8B. The small molecule is for example inhibiting receptors involved in BMP8B-dependend signaling. These receptors are for example selected from the group consisting of ALK3 receptor, ALK4 receptor, ALK5 receptor, ALK6 receptor, ALK7 receptor and combinations thereof. The small molecule BMP8B inhibitor is for example a pool of at least two individual small molecules. The small molecule or the pool of small molecules is for example used in a picomolar, nanomolar or micromolar range.

The BMP8B-inhibitor of the present invention is for example selected from the group consisting of oligonucleotide, antibody or fragment thereof, a small molecule or a combination thereof.

The BMP8B-inhibitor of the present invention inhibits expression and/or activity of BMP8B systemically, organ-specifically, tissue-specifically or cell-specifically. The BMP8B-inhibitor for example inhibits BMP8B expression and/or activity in the liver. The BMP8B-inhibitor for example inhibits BMP8B expression and/or activity in cells selected from the group consisting of hepatocyte, hepatic stellate cells, liver cancer cell or a combination thereof. The BMP8B-inhibitor for example inhibits expression and/or activity of BMP8B in the hepatocyte.

Hepatocytes represent the main cells of the parenchymal tissue of the liver and make up to 55 to 65% of the liver mass. HSC are pericytes found in the perisinusoidal space of the liver. HSC are the major cell type involved in liver fibrosis. Quiescent HSC represent 5 to 8% of the total number of liver cells in a normal and healthy liver.

Activation of hepatic stellate cell (HSC) represents the key event of liver fibrosis. Following liver injury, HSCs undergo an activation process to a highly proliferative, myofibroblast-like cell type and they are major source of extracellular matrix proteins in liver fibrosis (Hellerbrand, Pflugers Arch. 2013 June; 465(6):775-8. doi: 10.1007/s00424-012-1209-5. Epub 2013 Jan. 5. PMID: 23292551 Review 2013). For example HSC promote liver cancer likewise by inducing myeloid-derived suppressor cells through interleukin-6 signaling (Hellerbrand, Pflugers Arch. 2013 June; 465(6):775-8. doi: 10.1007/s00424-012-1209-5. Epub 2013 Jan. 5. PMID: 23292551 Review 2013; Filiol et al., Semin Liver Dis. 2019 July; 39(3):315-333. doi: 10.1055/s-0039-1685539. Epub 2019 Jun. 21).

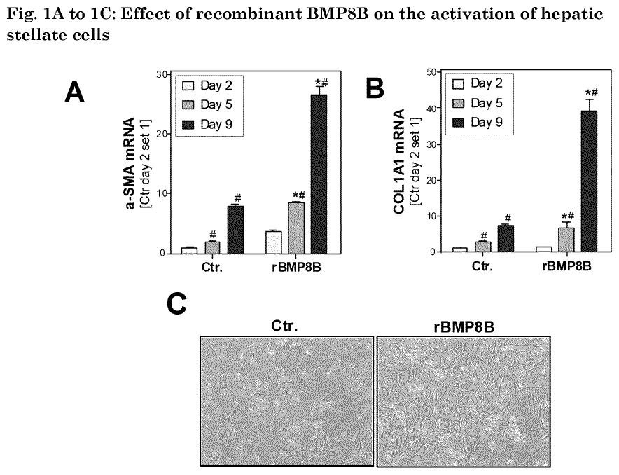

rBMP8B significantly promotes HSC activation. Alpha-smooth actin (α-SMA) and collagen type I (COL1A1) are established markers of HSC activation which expression is increased by BMP8B accordingly (see, FIGS. 1A and 1B).

Further, treatment with rBMP8B induces for example the phosphorylation of IkappaB-α and p65 as well as the expression of interleukin-8 (IL-8), intercellular adhesion molecule-1 (ICAM-1) and monocyte chemoattractant protein-1 (MCP-1) which are regulated by IkappaB-α. Both, the induction of phosphorylation of IkappaB-α as well as p65 and the induction of expression of interleukin-8 (IL-8), intercellular adhesion molecule-1 (ICAM-1) and monocyte chemoattractant protein-1 (MCP-1) is BMP8B dose-dependent. IkappaB-α, ICAM-1 and MCP-1 are known promoters of liver fibrosis.

The BMP8B-inhibitor of the present invention inhibits activity e.g., expression and/or phosphorylation, of markers of HSC activation. The BMP8B-inhibitor for example inhibits phosphorylation of IkappaB-α and/or p65. The BMP8B-inhibitor inhibits for example expression of genes selected from the group consisting of interleukin-8 (IL-8), intercellular adhesion molecule-1 (ICAM-1) and monocyte chemoattractant protein-1 (MCP-1).

Further, the present invention refers to a pharmaceutical composition comprising a BMP8-inhibitor of the present invention and a pharmaceutical acceptable carrier, excipient and/or dilutant. The pharmaceutical composition for example further comprises another active agent such as a chemotherapeutic, a multikanase-inhibitor for example sorafenib, a tyrosinekinase-inhibitor for example lenvatinib or regorafenib, an immune checkpoint-inhibitor for example nivolumab, obeticholic acid (OCA), or a combination thereof.

The BMP8B-inhibitor or a pharmaceutical composition comprising the BMP8B-inhibitor are used in a method for the prevention and/or treatment of liver disease such as liver fibrosis and/or liver cancer. Since liver fibrosis and liver cancer often appear in parallel, the BMP8B-inhibior advantageously is used in a method for the prevention and/or treatment of liver fibrosis and liver cancer in parallel for example in defines phases and defined pathological steps, respectively, which are characteristic for liver fibrosis or liver cancer.

Liver cancer is caused by FLD or any other disease or liver disease resulting in liver cancer.

The BMP8B-Inhibitor of the Present Invention for Use in Preventing and/or Treating Liver Fibrosis

The present invention, the BMP8B-inhibitor and/or a pharmaceutical composition comprising the BMP8B of the present invention is for use in a method of preventing and/or treating liver fibrosis.

The BMP8B-inhibitor or a pharmaceutical composition is administered locally and/or systemically for example orally, sublingually, nasally, subcutaneously, intravenously, intraperitoneally, intramuscularly, intrathecal, transdermal, intraarticular, intranasal, intrapleural, per inhalation, intraurethral and/or intra vesical. In addition, the BMP8B-inhibitor or a pharmaceutical composition of the present invention is for example used in ex vivo treatment of a transplant.

The BMP8B-inhibitor or a pharmaceutical composition of the present invention is administered one time or repetitively, e.g., once a day, once a week, once or twice a month, every three months, every six months for example for an unlimited time period from the diagnosis of liver fibrosis. According to the present invention, one or more inhibitors of the present invention can be administered together, at the same time point for example in a pharmaceutical composition or separately, or on staggered intervals.

Optionally the BMP8B-inhibitor for use in preventing and/or treating liver fibrosis is combined with any other compound or composition used in a method of preventing and/or treating liver fibrosis or radiotherapy.

Diagnosis of Liver Fibrosis by Measuring the Concentration of BMP8B

In addition, the present invention refers to a method for diagnosing liver fibrosis. Such method is used for example to identify a subject such as a patient who benefits from a BMP8B-inhibitor of the present invention used in a method for the treatment of liver fibrosis or to monitor the response of the subject to the use of the BMP8B-inhibitor of the present invention in a method for the prevention and/or treatment of liver fibrosis. For example the method for diagnosing liver fibrosis, e.g., the status of the liver fibrosis, is performed one time or in regular time periods such as once every 6 months or every 12 months. Optionally the method for diagnosing liver fibrosis is performed as a companion diagnostic.

Fibrosis is characterized by excessive production and deposition of extracellular matrix (ECM) proteins. The cellular source of the excessive ECM production are active hepatic stellate cells (HSC). BMP8B concentration is increased upon HSC activation whereas BMP8B is not detectable in samples of normal, healthy liver tissue. Hence, a detection or increase of the BMP8B concentration is a marker for diagnosing liver fibrosis.

Liver fibrosis is further characterized by a concentration of BMP8B in a cell, tissue, organ or sample of a subject being increased in comparison to the concentration of BMP8B in normal, healthy cell, tissue, organ or sample of a subject. The concentration of BMP8B is for example measured in a test sample and the concentration of the BMP8B is compared to the concentration of BMP8B of a healthy control sample, wherein an increase in the BMP8B concentration indicates liver fibrosis.

BMP8B is secreted by steatotic (living) hepatocytes and acts pro-fibrogenic and activating on HSC. Hence, measuring the concentration of BMP8B provides information about the interplay of lipid loaded hepatocytes and HSC, which activation and proliferation is the driver of fibrosis. Measuring the concentration of BMP8B therefore surprisingly allows to monitor the dynamic process of ECM production, HSC proliferation and ultimately fibrosis progression. Accordingly, an increased BMP8B concentration correlates with a high or increased degree of fibrosis Several scales for staging and grading of liver fibrosis exist for monitoring and evaluating the degree of fibrosis and its progression. Liver fibrosis grading systems for example comprise the Ishak score (0-6 stages), the Knodell (0-4 stages) score and the Metavir score (0-4 stages). Healthy normal liver is attributed as stage 0 in all systems, and an increase of fibrogenic tissue is monitored by ascending stages. For example, the Metavir score is measuring both activity score (A), A0 to A3, indicating the activity of inflammation in the liver and fibrosis score (F), F0 to F4, representing amount of fibrosis and/or scaring and allowing to monitor the degree of fibrosis as shown in the following Table 1:

| Activity score (A) | Fibrosis score (F) |
|---|---|
| A0: No activity | F0: No fibrosis |
| A1: Mild activity | F1: Portal fibrosis without |
| | septa/mild fibrosis |
| A2: Moderate activity | F2: Portal fibrosis with few |
| | septa/moderate fibrosis |
| A3: Severe activity | F3: Numerous septa |
| | without cirrhosis/severe |
| | fibrosis |
| | F4: Cirrhosis |

For diagnosing liver diagnosis and its degree it is particularly important to discriminate between F0 and F1, i.e., to diagnose the onset of fibrosis, and in addition or alternatively, to discriminate between F0-F1 and F2-F4, i.e., to diagnose the presence of relevant fibrosis and to discriminate between F0-F2 and F3-F4, i.e., to diagnose the progression of fibrosis towards advanced fibrosis.

BMP8B concentrations according to measurements of the present invention significantly differ in test samples of subjects having no fibrosis (F0), portal fibrosis without septa/mild fibrosis (F1), portal fibrosis with few septa/moderate fibrosis (F2), numerous septa without cirrhosis/severe fibrosis (F3) and cirrhosis (F4). Hence, the present invention comprises a method for monitoring and/or diagnosing liver fibrosis, wherein an increase of the concentration of BMP8B in a cell, tissue, organ or sample of a subject correlates with the degree of the liver fibrosis.

For example the increase of the BMP8B concentration distinguishes between fibrosis stages according to fibrosis staging scales. For example the increase of the BMP8B concentration distinguishes between stage F0 to F4 according to the Metavir score. For example the increase of the BMP8B concentration distinguishes between (i) stage F0 and F1, F2, F3 or F4 and/or (ii) between stage F0, F1 or F2 and F3 or F4.

Further, the method for diagnosing liver fibrosis and/or the degree of liver fibrosis according to the present invention is performed one time or in regular time periods such as once every 6 months or every 12 months.

Moreover, the method for diagnosing liver fibrosis and/or the degree of liver fibrosis is for example performed together with the measurement of the concentration of one or more additional marker(s) indicative of liver fibrosis, for example at the same time point or separately, or on staggered intervals. Such marker is for example selected from the group consisting of M30, a N-terminal peptide (PIIINP), a tissue inhibitor of metalloproteinase 1 (TIMP-1) or a combination thereof.

The sample according to the present invention is selected from the group consisting of blood sample, serum sample, plasma sample, salvia sample, urine sample, tissue sample, or a combination thereof. The sample is for example a blood and/or tissue sample.

The BMP8B concentration is measured by any technique known to a person skilled in the art. For example the BMP8B concentration is measured by a technique selected from the group consisting of immunohistochemistry, western blot, immunoblotting, quantitative real time PCR, two-dimensional-electrophoresis, proteomics, chromatography, colorimetry, enzyme-linked immunosorbent assay (ELISA), QuantiGene or combinations thereof.

The BMP8B-Inhibitor of the Present Invention is for Use in Preventing and/or Treating Liver Cancer.

The BMP8B-inhibitor of the present invention or a pharmaceutical composition comprising the BMP8B-inhibitor is also for use in a method of preventing and/or treating liver cancer.

The BMP8B-inhibitor or a pharmaceutical composition is administered locally and/or systemically, for example orally, sublingually, nasally, subcutaneously, intravenously, intraperitoneally, intramuscularly, intratumoral, intrathecal, transdermal, intraarticular, intranasal, intrapleural, per inhalation, intraurethral and/or intra vesical.

Further, the BMP8B-inhibitor or a pharmaceutical composition of the present invention is administered one time or e.g., once a day, once a week, once or twice a month, every three months, every six months for example for an unlimited time period from the diagnosis of liver cancer. According to the present invention, one or more inhibitors of the present invention can be administered together, at the same time point for example in a pharmaceutical composition or separately, or on staggered intervals.

Optionally the BMP8B-inhibitor for use in a method of preventing and/or treating liver cancer is combined with any other compound or composition used in a method of preventing and/or treating liver cancer or radiotherapy.

Diagnosis of Liver Cancer by Measuring the Concentration of BMP8B

In addition, the present invention refers to methods for diagnosing liver cancer wherein such method can be used for example to identify a subject such as a patient who benefits from a BMP8B-inhibitor of the present invention used in the treatment of liver cancer or to monitor the response of the subject to the use of the BMP8B-inhibitor of the present invention in the prevention and/or treatment of liver cancer. For example the method for diagnosing liver cancer, e.g., the status of the liver cancer, is performed one time or in regular time periods such as once every 6 months or every 12 months. Optionally the method for diagnosing liver cancer is performed as a companion diagnostic.

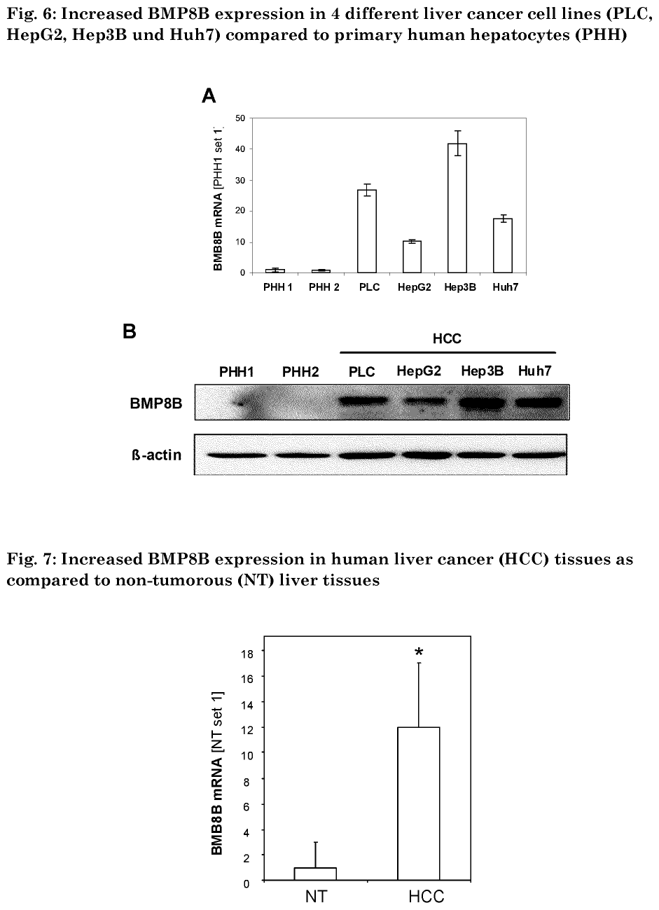

Concentration is significantly increased in human liver cancer cell lines and liver cancer tissues as compared to primary human hepatocytes and non-tumorous liver tissues where BMP8B is not detectable (see, FIG. 6 and FIG. 7). BMP8B acts pro-tumorigenic on liver cancer cells. Hence, the detection of BMP8B concentration is a marker for diagnosing liver cancer.

The present invention also refers to a method for diagnosing liver cancer. Liver cancer is characterized by BMP8B concentration in a cell, tissue, organ or sample of a subject being increased in comparison to the concentration of BMP8B in normal, healthy cell, tissue, organ or a sample of a healthy subject. The concentration of BMP8B is for example measured in a test sample and the concentration of the BMP8B is compared to the concentration of BMP8B of a healthy control sample, wherein an increase in the BMP8B concentration indicates liver cancer.

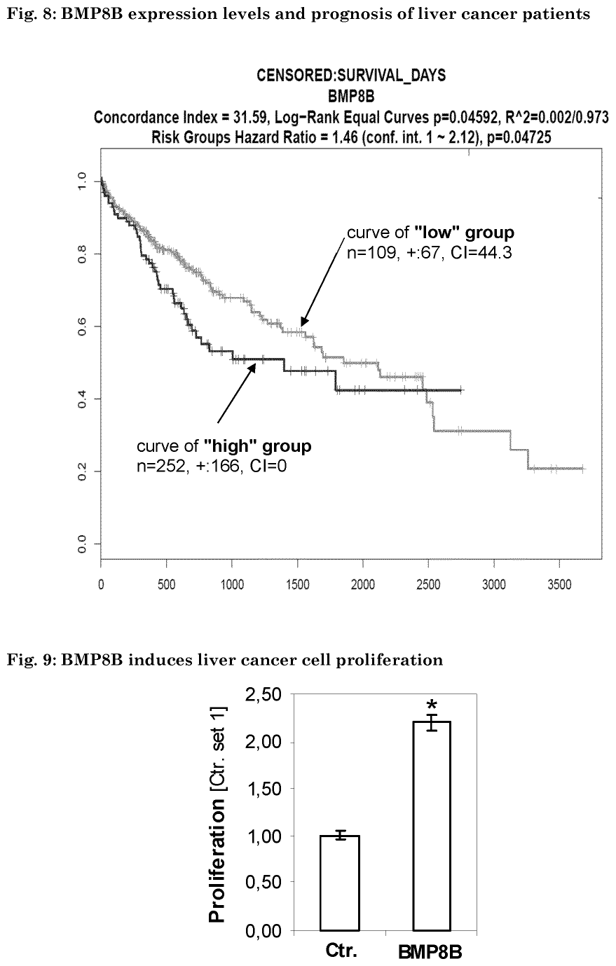

High concentration of BMP8B correlates with poor survival rates of patients suffering from liver cancer (FIG. 8). These results indicate that BMP8B concentrations correlate with the degree and/or progression of liver cancer likewise, allowing to monitor the stage of liver cancer based on BMP8B concentration.

The present invention comprises a method for monitoring and/or diagnosing liver cancer, wherein an increase of the concentration of BMP8B in a cell, tissue, organ or sample of a subject correlates with the degree and/or stage of liver cancer. High concentration of BMP8B indicates for example fast cancer progression and/or correlates for example with a late stage of the liver tumor (e.g., stage 3 or 4).

Further, the method for diagnosing liver cancer and/or the degree of liver cancer and/or the progression of liver cancer according to the present invention is performed one time or in regular time periods such as once every 6 months or every 12 months.

The method for diagnosing liver cancer and/or the degree of liver cancer and/or the progression of liver cancer is for example performed together with the measurement of the concentration of one or more additional marker(s) indicative of liver cancer, for example at the same time point or separately, or on staggered intervals. An additional marker is for example selected from the group consisting of total alpha-fetoprotein (AFP), Lens culinaris agglutinin-reactive AFP (AFP-L3), protein induced by vitamin K absence or antagonist-II (PIVKA-II) or combinations thereof.

The sample according to the present invention is selected from the group consisting of blood sample, serum sample, plasma sample, salvia sample, urine sample, tissue sample, or a combination thereof. The sample is for example a blood and/or tissue sample.

The BMP8B concentration is measured by any technique known to a person skilled in the art. For example the BMP8B concentration is measured by a technique selected from the group consisting of immunohistochemistry, western blot, immunoblotting, quantitative real time PCR, two-dimensional-electrophoresis, proteomics, chromatography, colorimetry, enzyme-linked immunosorbent assay (ELISA), QuantiGene or combinations thereof.

The present invention also refers to the use of a BMP8B-inhibitor or a pharmaceutical composition comprising the BMP8B-inhibitor of the present invention for treating and/or preventing liver fibrosis and/or liver cancer which has previously been diagnosed with the method of diagnosing liver fibrosis and/or liver cancer according to the present invention. Treatment, prevention and diagnosis according to the present invention is for example performed in parallel, alternatingly or sequentially one time or multiple times during the period of the disease, i.e., liver fibrosis and/or liver cancer.

The combination of diagnosis and use of the BMP8B-inhibitor in the prevention and/or treatment enables quick onset of the treatment, precise monitoring of the treatment and optionally parallel treatment of liver fibrosis and liver cancer.

The present invention is further directed to a kit for diagnosing liver fibrosis and/or liver cancer in a method according to the present invention. The kit comprises or consists of a reagent that specifically detects BMP8B concentration and optionally additional reagents for the detection of the presence and/or concentration of one or more additional marker(s) indicative of liver fibrosis and/or liver cancer. Such marker are for example M30, N-terminal peptide (PIIINP), or tissue inhibitor of metalloproteinase 1 (TIMP-1), alpha-fetoprotein (AFP), Lens culinaris agglutinin-reactive AFP (AFP-L3), protein induced by vitamin K absence, e.g., for liver fibrosis, or antagonist-II (PIVKA-II) for liver cancer. The reagent of the kit of the present invention comprises for example an antibody or fragment thereof, a probe, a primer or a combination thereof for the detection of the BMP8B concentration in the test sample.

The sample according to the present invention is selected from the group consisting of blood sample, serum sample, plasma sample, salvia sample, urine sample, tissue sample, or a combination thereof. The sample is for example a blood and/or tissue sample.

Optionally the kit may further comprise instructions for use of the kit and/or interpretation of the measurements obtained by the kit. Optionally the kit also comprises a control sample for comparison of the measured BMP8B concentration.

A subject of the present invention is for example a mammalian such as a human, cat, dog or horse, a bird or a fish.

### EXAMPLES

The following examples show the present invention in more detail, however, the invention is not limited to these examples.

### Example 1: Effect of BMP8B on Hepatic Stellate Cells

Hepatic stellate cells (HSC) are liver-specific pericytes within the vasculature of the hepatic sinusoid. Under physiological conditions, HSCs reside in a quiescent stage. Following liver injury, HSCs undergo an activation process to a highly proliferative, myofibroblast-like cell type and they are the major source of extracellular matrix proteins in liver fibrosis (Hellerbrand, Pflugers Arch. 2013 June; 465(6):775-8. doi: 10.1007/s00424-012-1209-5, 2013). Therefore, the activation of HSC is the key event of hepatic fibrosis. Also in NAFLD fibrosis, activated HSC are the major cellular source of extracellular matrix deposition.

The activation process of HSC can be simulated in an established in vitro model. Primary human hepatic stellate cells (HSC) were isolated and activated by culturing on plastic dishes for 9 days with or without recombinant BMP8B-treatment (doses up to 10 nM). Here, freshly isolated primary (quiescent) HSC undergo within approx. 8-10 days the same pathophysiological changes as in vivo.

In this model, rBMP8B significantly accelerated the HSC-activation process. FIG. 1A depicts the accelerated increase of alpha-smooth muscle actin (α-SMA) expression, which is recognized as a marker of activated HSCs.

The effect of recombinant BMP8B (rBMP8B) on the activation of HSC has been analyzed in this model. One day after isolation, primary human HSC were incubated with rBMP8B (10 nM). After 2, 5 and 9 days, expression levels of alpha-smooth muscle actin (α-SMA) and collagen type I (COL1A1) were determined by quantitative RT-PCR analysis. α-SMA and collagen type I expression increases during the activation of HSC and thus are established markers of HSC activation. Treatment with rBMP8B significantly enhanced the expression of both markers compared to control HSC (FIGS. 1A and 1B) and also microscopic phase-contrast images of cultured HSC at day 9 indicated that rBMP8B induced the in vitro transforming process of HSC towards an activated myofibroblast like cell type (FIG. 1C). (*: p<0.05 compared with corresponding condition w/o BMP8B stimulation; #: p<0.05 compared with untreated control day 2). This data show, that BMP8B induces the activation of hepatic stellate cells.

### Example 2: Effect of Recombinant BMP8B on Proliferation of Activated Hepatic Stellate Cells

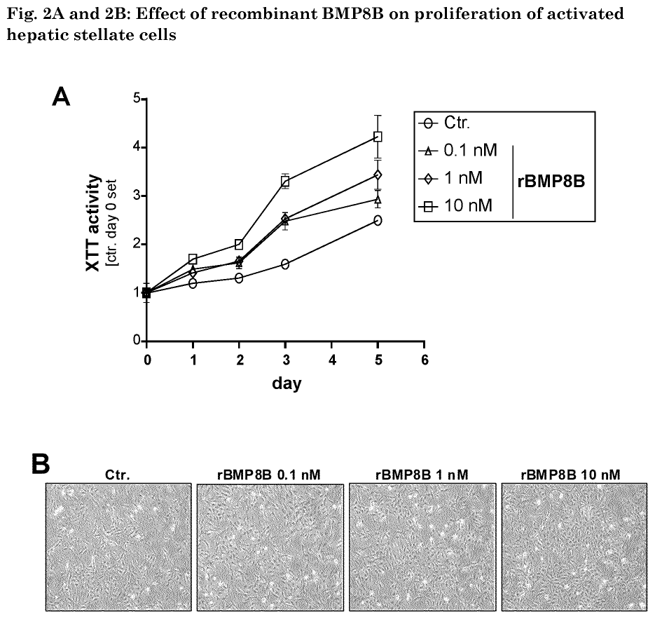

Once HSC are activated, the proliferation rate of HSC significantly increases, a mechanism that has been proposed to play a crucial role in the progression of fibrosis in chronic liver disease including NAFLD. Incubation of activated human HSC with rBMP8B led to dose-dependent induction of proliferation (FIGS. 2A and 2B). Activated HSC were treated with different doses of recombinant BMP8B. Proliferation were assessed with the colorimetric XTT assay and phase-contrast images of activated HSC were taken at 5 days after incubation. (*: p<0.05 compared to control).

### Example 3: Effect of Recombinant BMP8B on Activation of Pro-Fibrogenic Signaling Pathways and Pro-Fibrogenic Gene Expression

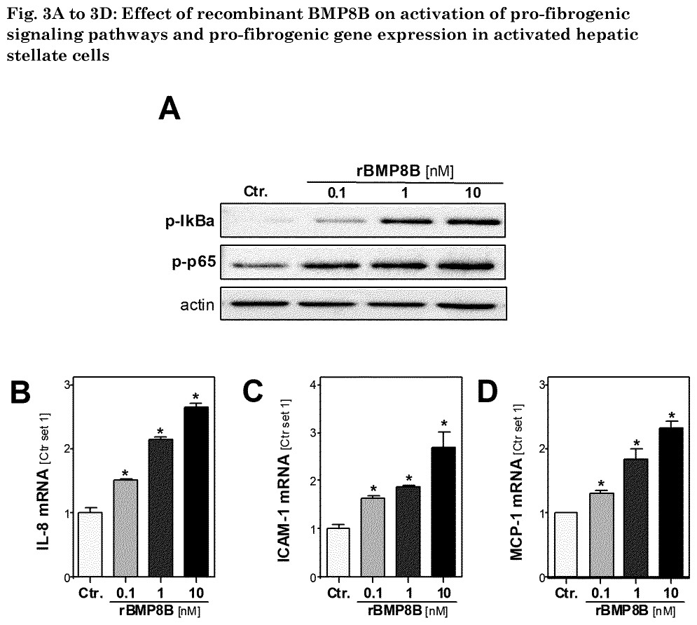

Moreover, treatment of activated HSC with rBMP8B led to a dose-dependent induction of the phosphorylation of IkappaB-α and p65. Western blot analysis of p-p65 and p-IκBa protein levels; actin served as control for loading adjustment (FIG. 3A). Fitting to this, treatment of activated HSC with rBMP8B dose dependently induced the expression of interleukin-8 (IL-8), intercellular adhesion molecule-1 (ICAM-1) and monocyte chemoattractant protein-1 (MCP-1) (FIG. 3B-D), which are known to be regulated by NFkappaB. It has been shown that NFkappaB in HSC as well ICAM-1 and MCP-1 are promotors of hepatic fibrosis. Cellular mRNA levels of IL-8, ICAM-1 and MCP-1 were analyzed by quantitative RT-PCR. (*: p<0.05 compared to control).

FIG. 1 to 3 show that recombinant BMP8B treatment significantly enhanced the activation of hepatic stellate cells (HSC) as well as the pro-fibrogenic phenotype of already activated HSC, indicating a direct pro-fibrogenic effect of BMP8B on the key-mediator cells of hepatic fibrosis.

### Example 4: BMP8B Depletion in Primary Human Hepatocytes

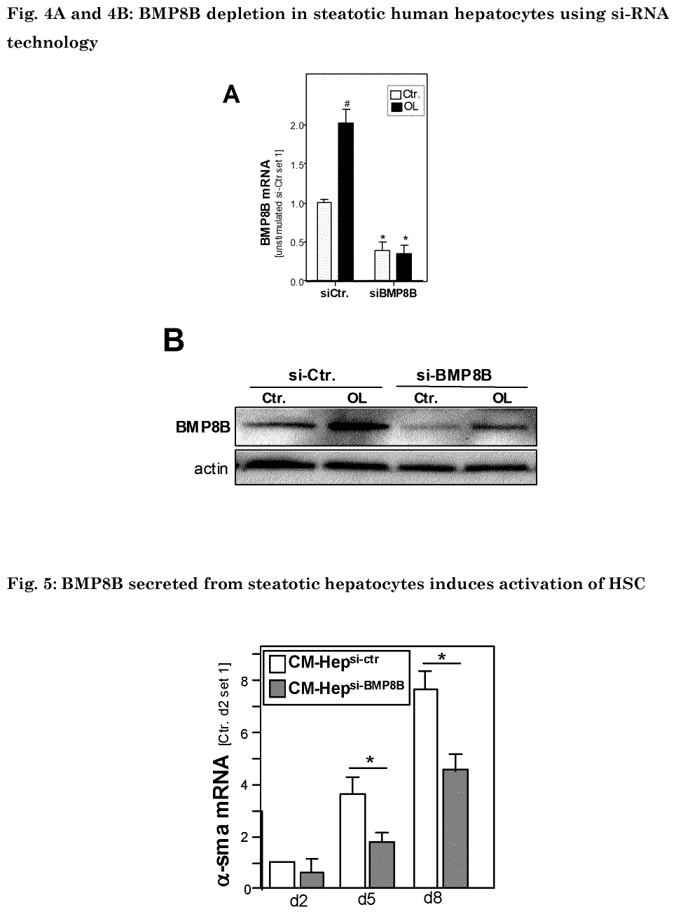

It has previously been shown that lipid accumulation in hepatocytes induces BMP8B expression. Applying siRNA technology (transfection with siRNA pools) leads to significant downregulation of the BMP8B expression in control hepatocytes and to almost completely abrogate the steatosis induced BMP8B expression (FIGS. 4A and 4B). BMP8B expression was depleted in primary human hepatocytes by transfection with si-RNA pools directed against BMP8B(si-BMP8B) and control cells were transfected with control-siRNA pools (si-Ctr). Subsequently, cells were incubated with or without 0.4 mM oleate (OL) for additional 24 h. BMP8B mRNA levels are analyzed by quantitative RT-PCR and BMP8B protein levels are analyzed by Western blot; (*: p<0.05 compared with si-Ctr transfected cells; #: p<0.05 compared with cells w/o oleate stimulation).

### Example 5: BMP8B Secreted from Steatotic Hepatocytes Induces Activation of HSC

Further, freshly isolated primary human hepatic stellate cells (HSC) have been incubated with conditioned media (CM) generated from BMP8B depleted primary human hepatocytes (CM-Hep si-BMP8B) and control hepatocytes (CM-Hep si-Ctr) and the activation process of the HSC has been subsequently monitored (FIG. 5). After day 5 and day 8 the α-SMA expression was significantly lower the presence of CM-Hep si-Ctr compared with CM-Hep si-BMP8B. This once indicates, that BMP8B secreted from steatotic hepatocytes induces the activation of HSC. Primary human hepatic stellate cells (HSC) were isolated and activated by culturing on plastic dishes for 8 days with conditioned media (CM) from BMP8B depleted primary human hepatocytes (CM-Hep si-BMP8B) and control hepatocytes (CM-Hep si-Ctr). α-SMA mRNA levels were analyzed by quantitative RT-PCR. α-SMA is a marker for HSC-activation that increases with time. (*: p<0.05).

Data of FIGS. 4 and 5 show that the pro-fibrogenic, steatosis induced BMP8B expression in human hepatocytes can successfully inhibited with si-pools directed against BMP8B.

### Example 6: Increased BMP8B Expression in 4 Different Liver Cancer Cell Lines (PLC, HepG2, Hep3B Und Huh7) Compared to Primary Human Hepatocytes (PHH)

BMP8B expression is significantly upregulated in human liver cancer cell lines compared with primary human hepatocytes. BMP8B mRNA levels were analyzed be quantitative RT-PCR (FIG. 6A) and BMP8B protein levels (FIG. 6B) are analyzed by Western blot.

### Example 7: Increased BMP8B Expression in Human Liver Cancer (HCC) Tissues as Compared with Non-Tumorous (NT) Liver Tissues

BMP8B expression is significantly upregulated in human liver cancer (HCC) tissues as compared with non-tumorous (NT) liver tissues. Analysis of BMP8B mRNA expression in liver cancer (HCC) and corresponding NT tissues of 10 liver cancer patients shown in FIG. 7. BMP8B levels were analyzed by quantitative RT-PCR (*: p<0.05).

### Experiment 8: BMP8B Expression Levels and Prognosis of Liver Cancer Patients

High expression of BMP8B in liver cancer tissues correlates with poor patient survival. Kaplan-Meier survival curve analysis using SurvExpress Biomarker validation database (Aguirre-Gamboa, et al. SurvExpress: An online biomarker validation tool and database for cancer gene expression data using survival analysis. PLoS ONE 2013, 8, e74250.) for a TCGA HCC (LIHC) dataset (n=361) for overall survival with stratification into ‘lowrisk’ and ‘high-risk’ groups based on the prognostic index. The analysis of this dataset revealed a reduced overall survival in liver cancer patients with high BMP8B expression compared to liver cancer patients with low BMP8B expression and is shown in FIG. 8.

### Example 9: BMP8B Induces Liver Cancer Cell Proliferation

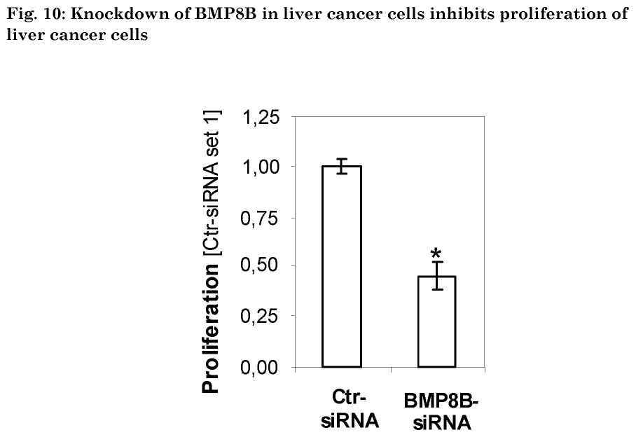

Stimulation with recombinant BMP8B protein induces liver cancer cell proliferation. Human liver cancer cells (HepG2) were stimulated with recombinant BMP8B (10 nM) for 2 days. XTT-assay was used to determine proliferation (*: p<0.05) as shown in FIG. 9.

### Example 10: Knockdown of BMP8B in Liver Cancer Cells Inhibits Proliferation of Liver Cancer Cells

Knockdown of BMP8B in liver cancer cells using BMP8B-directed siRNA inhibits liver cancer cell proliferation. Human liver cancer cells (HepG2) were transfected with siRNA directed against BMP8B or Ctr-siRNA. XTT-assay was used to determine cell proliferation (*: p<0.05) as shown in FIG. 10.

FIG. 6 to 10 show that BMP8B acts pro-tumorigenic in liver cancer cells, that BMP8B effects can be paracrine (coming from steatotic hepatocytes) or autocrine (coming from the liver cancer (HCC) cells themselves) and that BMP8B levels (in serum or tissue) can act as a diagnostic marker for both risk of liver cancer development and liver cancer progression.

1. BMP8B-inhibitor for use in a method of preventing and/or treating liver fibrosis and/or liver cancer, wherein the inhibitor is an oligonucleotide, an antibody or fragment thereof, a small molecule or a combination thereof.

2. BMP8B-inhibitor for use according to claim 1, wherein the oligonucleotide is siRNA, miRNA, an antisense oligonucleotide or a combination thereof.

3. BMP8B-inhibitor for use according to claim 1 or 2, wherein the siRNA is one type of siRNA or a pool of siRNAs.

4. BMP8B-inhibitor for use according to claim 3, wherein the siRNA or the pool of siRNAs is in the picomolar or nanomolar range.

5. BMP8B-inhibitor for use according to any one of claims 1 to 4, wherein expression and/or activity of the BMP8B is inhibited in the hepatocyte.

6. BMP8B-inhibitor for use according to any one of claims 1 to 5, wherein induction of phosphorylation of IkappaB-α and/or p65, the expression of interleukin-8 (IL-8), of intercellular adhesion molecule-1 (ICAM-1), of monocyte chemoattractant protein-1 (MCP-1) or a combination thereof is inhibited.

7. BMP8B-inhibitor for use according to any one of claims 1 to 6, wherein the BMP8B-inhibitor is administered locally or systemically.

8. Pharmaceutical composition comprising a BMP8B-inhibitor for use according to any one of claims 1 to 7 and a pharmaceutically acceptable carrier.

9. Method for diagnosing liver fibrosis, wherein the concentration of BMP8B is measured in a test sample and the concentration of the BMP8B is compared to the concentration of BMP8B of a healthy control sample, wherein an increase in the BMP8B concentration indicates liver fibrosis.

10. Method according to claim 9, to identify a subject that will benefit from the use of the BMP8B-inhibitor according to any one of claims 1 to 7 or to monitor the response of the subject to the use of the BMP8B-inhibitor according to any one of claims 1 to 7.

11. Method according to claim 9 or 10, wherein the increase of the concentration of BMP8B measured in the sample correlates with the degree of the liver fibrosis for example the increase of the BMP8B concentration distinguishes (i) between stage F0 and F1, F2, F3 or F4, or (ii) between stage F0, F1 or F2 and F3 or F4, or (iii) between stage F0 or F1 and F2, F3 or F4.

12. Method according to any one of claims 9 to 11, wherein the sample is a blood sample or a tissue sample.

13. Method according to any one of claims 9 to 12, wherein the technique for measuring the BMP8B concentration in the sample is selected from the group consisting of two-dimensional electrophoresis, proteomics, chromatography, Western Blot, immunoblotting, enzyme-linked immunosorbent assay (ELISA) or a combination thereof.

14. Method according to any one of claims 9 to 13, wherein the concentration of one or more additional marker(s) indicative of liver fibrosis is measured.

15. Kit for diagnosing liver fibrosis or liver cancer in a method according to any one of claims 9 to 14, wherein the kit comprises an antibody or fragment thereof, a probe, a primer or a combination thereof, for the detection of the BMP8B concentration and optionally for the detection of the presence and/or concentration of one or more additional marker(s) indicative of liver fibrosis or liver cancer.
